(19) United States
(12) Patent Application Publication
(10) Pub. No.: US 2006/0041265 A1
(43) Pub. Date: Feb. 23, 2006

(54) Shell for scalpel

(76) Inventor: Howard L. Shackelford SR., Triadelphia, WV (US)

Correspondence Address: David C. Jenkins;Eckert Seamans Cherin & Mellott, LLC, 44th Floor, 600 Grant Street, Pittsburgh, PA, 15219, US

(21) Appl. No.: 10/920,084

(22) Filed: Aug. 17, 2004

(51) Int. Cl. A61B 17/32

(52) U.S. Cl. 606/167

(57) ABSTRACT

A jacket for a scalpel id provided. A disposable scalpel has an elongated body with a base wall and two sidewalls forming a scalpel channel, and a blade assembly having a blade coupled to a slider, the slider structured to move the blade between a first, retracted position and second, extended position. The jacket includes a body having an elongated base wall and two sidewalls. Each sidewall extends generally perpendicular to the jacket base wall, thereby defining a jacket channel. The jacket sidewalls are spaced apart a sufficient distance so that the scalpel may be disposed between the jacket sidewalls. The jacket further includes a trap structured to hold the scalpel in the jacket channel.

### BACKGROUND OF THE INVENTION

1. Field of the Invention

This invention relates to scalpels and, more specifically, to a jacket for a scalpel structured to provide added weight and/or length to a disposable scalpel.

2. Background Information

The use of disposable scalpels is increasing in many hospitals. The disposable scalpel is, typically, made from plastic, which forms a body having a channel portion and often having a handle portion. See, U.S. Pat. No. 6,254,621. A blade is disposed on a slider in the channel portion. The blade may be moved between a first, retracted position and a second, extended position. Some scalpels include a locked position wherein the blade is secured prior to disposal. Such disposable scalpels are inexpensive to manufacture and, because the scalpels are disposable, do not have to be sterilized after use.

The disadvantage to such scalpels is that disposable scalpels lack heft. That is, traditional metal scalpels, which were typically made from stainless steel, had a certain weight and balance. Physicians who have been trained with traditional metal scalpels tend to dislike the lighter disposable scalpels. Additionally, even users who have been trained using disposable scalpels prefer a scalpel with more heft as such scalpels provide, in a subjective sense, more control and feedback. That is, some users can more easily determine the location and orientation of a scalpel based on how the scalpel feels in their hand and that feeling is more intense with a heavier scalpel. Additionally, disposable scalpels tend to be manufactured at a standard length of about six inches. On certain occasions, a user may desire a scalpel having an extended length, for example, when performing certain thoracic procedures. Because the need for extended length scalpels is less common, disposable extended length scalpels are more expensive.

There is, therefore, a need for a jacket structured to be coupled to a disposable scalpel and structured to add heft.

There is a further need for a jacket structured to be coupled to a disposable scalpel having an extended length.

There is a further need for a jacket structured to be coupled to existing disposable scalpels.

### SUMMARY OF THE INVENTION

These needs, and others, are met by the present invention which provides a jacket structured to be coupled to a disposable scalpel. The jacket is weighted and/or has an extended length. The jacket includes a body forming a channel. The channel is slightly larger than the scalpel and is structured to snuggly engage the scalpel. The jacket may be made from a heavier metal, such as, but not limited to, steel, copper, brass, silver, or gold. As such, the jacket may be sterilized and used repeatedly. Alternatively, the jacket may be made from plastic having a weighted material disposed therein. For example, the plastic may be made with weighted particles, such as, but not limited to, sand or steel mixed into the plastic matrix. The weighted material may also be a strip of material, for example, but not limited to, a steel bar encased in the plastic. Additionally, by altering the dimensions of the body, the weight and balance of the jacket may be adjusted. That is, the jacket may be made to be front or back heavy, or balanced. In one embodiment, the jacket is slightly larger than the scalpel. Alternatively, the jacket may include an extended handle portion which, when a scalpel is disposed in the jacket, effectively extends the length of the scalpel.

It is an object of this invention to provide a jacket structured to be coupled to a disposable scalpel and structured to add heft.

It is a further object of this invention to provide a jacket structured to be coupled to a disposable scalpel having an extended length.

It is a further object of this invention to provide a jacket structured to be coupled to existing disposable scalpels.

### BRIEF DESCRIPTION OF THE DRAWINGS

A full understanding of the invention can be gained from the following description of the preferred embodiments when read in conjunction with the accompanying drawings in which:

FIG. 1 is an isometric view of a scalpel jacket and a disposable scalpel.

FIG. 2 is an isometric view of an alternate scalpel.

FIG. 3 is a detailed view of an alternate trapping structure.

FIG. 4 is a detailed view of a scalpel jacket with an alternate trapping structure.

FIG. 5 is an isometric view of a scalpel jacket with an alternate trapping structure.

FIG. 6 is an isometric view of a scalpel jacket with an alternate trapping structure.

FIG. 7 is a front view of an alternative scalpel and a scalpel jacket with an alternate trapping structure.

FIG. 8 is a side view of the jacket from FIG. 7.

### DESCRIPTION OF THE PREFERRED EMBODIMENTS

Directional phrases used herein, such as upper, lower, front, back, etc., relate to the orientation of the elements shown in the drawings and are not limiting upon the claims. It is further noted that the “forward” end of the scalpel 10 and the jacket 50 is the end from which the blade 30 extends when in the forward position.

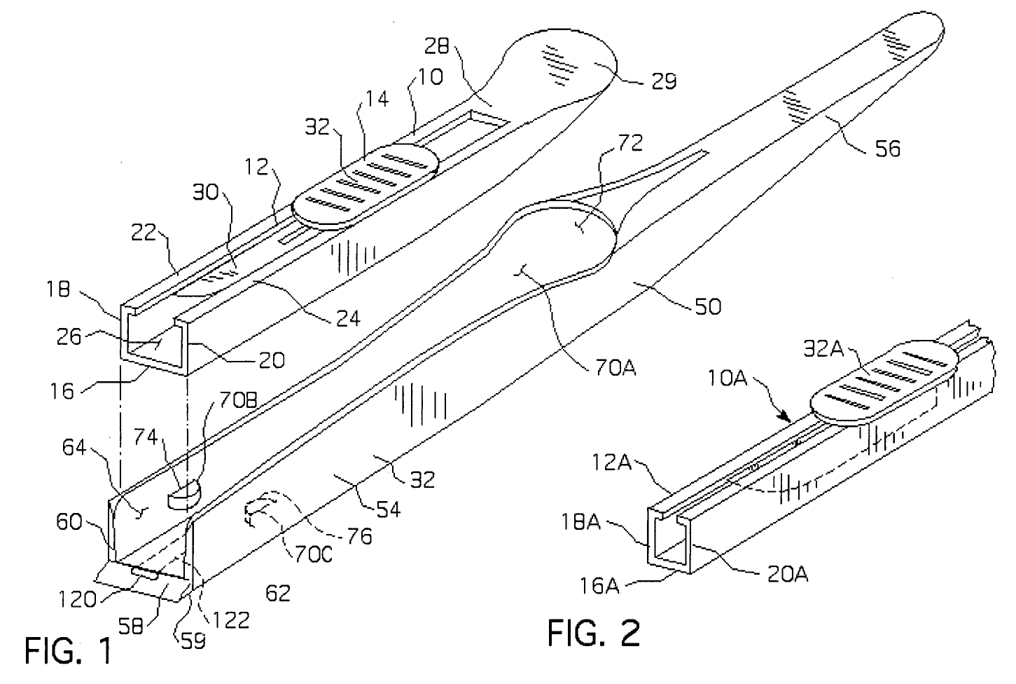

As shown in FIG. 1, a scalpel assembly 9 includes a scalpel 10 and a jacket 50. The scalpel 10 generally includes an elongated body 12 and a blade assembly 14. The body 12 has a base wall 16 and two generally perpendicular and peripheral sidewalls 18, 20. The sidewalls 18, 20 typically have inwardly extending flanges 22, 24. This configuration creates a channel 26. The channel 26 may extend over the length of the body 12, or, one end of the body 12 may be a solid handle portion 28. The scalpel handle portion 28 may have a flared section 29 so that the handle portion 28 is wider than the channel 26. The blade assembly 14 includes a blade 30 and a slider 32. The blade 30 is coupled to the slider 32. The slider 32 is structured to be movably coupled to the body 12 within the channel 26. Thus, the blade 30 may be moved between a first, retracted position and a second, extended position by moving the slider 32 within the channel 26. Additionally, some scalpels 10 include a locked position for the slider 32 so that the scalpel 10 may be disposed. As shown in the figures, the slider 32 is disposed on the “front” side of the body 12. Some scalpels 10A include the slider 32A extending from the “top” side of the body 12A. That is, as shown in FIG. 2, the base wall 16A is narrow and the sidewalls 18A, 20A have an extended length as compared to the base wall 16 and sidewalls 18, 20 of the scalpel 10 shown in FIG. 1. It is understood that the jacket 50, described below, is structured to engage one or the other type of scalpel 10, 10A. That is, the jacket 50 may have, for example, either a wide or narrow base wall 58 to correspond to the type of scalpel 10, 10A to be engaged.

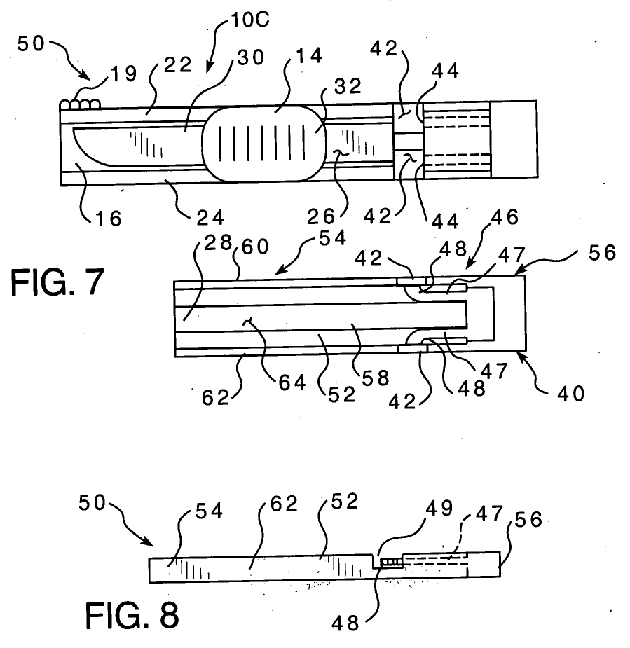

The jacket 50 includes an elongated body 52 having a channel portion 54 and a handle portion 56. The jacket channel portion 54 includes a base wall 58 and two sidewalls 60, 62. The jacket base wall 58 is sized to be, generally, the width of the scalpel base wall 16 and two sidewalls 18, 20. That is, the jacket base wall 58 is generally as wide as the entire scalpel 10. The jacket sidewalls 60, 62 extend generally perpendicularly from the peripheral edges of the jacket base wall 58, thereby forming a channel 64. Thus, the jacket channel portion 54 is sized to fit around the scalpel 10. Additionally, the jacket channel portion 54 may be sized, or, more specifically, have a length, that is structured to allow a portion of the scalpel 10 to extend from the jacket 50. That is, a scalpel 10 may have certain structures, for example, a saw-like grip portion 19 (FIG. 7) or a slider release button, disposed at the front end of the scalpel channel 26. Such scalpel 10 structures should not be covered by the jacket 50. By limiting the length of the jacket channel portion 54, the jacket 50 is structured so that the front end of the scalpel 10 extends from the jacket 50 as shown in FIG. 7. Additionally, the front edge of the jacket base wall 58 may include a rolled or angled flange 59. The rolled flange 59 allows the user to more easily flex the jacket 50 during insertion and removal of the scalpel 10.

The jacket handle portion 56 is, typically, a solid, elongated member having outer cross-sectional dimensions generally corresponding to the outer cross-sectional dimensions of the channel portion 54. The handle portion 56 may be relatively short, e.g., extending about 0.25 inch to about 1.0 inch beyond the channel portion 54. Such a short handle portion 56 is not intended to add length to the overall scalpel/jacket combination and is instead structured to add only the additional weight as described below. Conversely, the handle portion 56 may also have an extended length, typically more than about 1.0 inch to about 6.0 inches, beyond the channel portion 54. In this embodiment, the jacket 50 effectively adds length to a scalpel 10.

The jacket 50 further includes a trap 70 structured to retain the scalpel 10 within the jacket channel portion 54. Three trap 70 structures are shown in FIG. 1. While multiple trap 70 structures may be used, a jacket 50 typically has only one trap 70. A first trap 70A is a shaped portion 72 of the jacket channel 64 that is structured to correspond to the shape of a scalpel handle portion flared section 29. Preferably, the jacket channel shaped portion 72 is a tight fit about the scalpel 10 thereby creating a snap-fit. A second trap 70B includes one or more protrusions 74 that extend from a jacket sidewall 60, 62 into the jacket channel 64. As the jacket channel 64 is structured to fit snugly about the scalpel 10, the one or more protrusions 74 engage the scalpel 10 in a snap-fit manner. Alternatively, the protrusion 74 may be structured to flex and bias said scalpel 10 against the opposite sidewall 60, 62. A third trap 70C includes a post 76 extending from the jacket base wall 58 into the jacket channel 64. Typically, disposable scalpels 10 are molded with an opening (not shown) in the scalpel base wall 16. The post 76 is structured to fit within the scalpel opening. Thus, when the scalpel 10 is disposed within the jacket 50 and the post 76 is disposed within the opening, the scalpel 10 is prevented from sliding forward in the jacket 50.

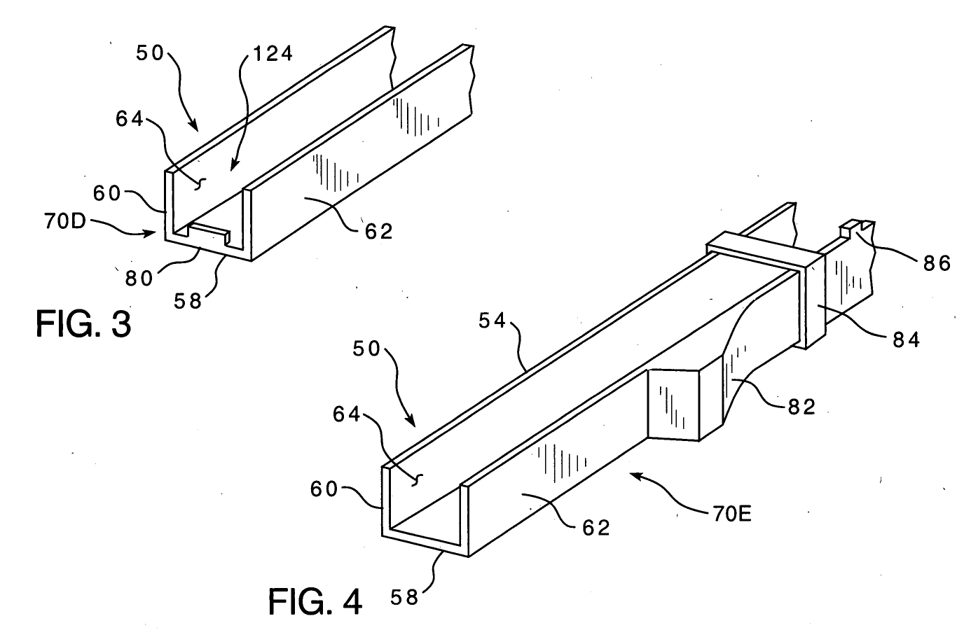

As shown in FIG. 3, a fourth type of trap 70D is a tab 80 disposed at the front end of the jacket channel 64. The tab 80 extends from the jacket base wall 58 generally perpendicularly into the jacket channel 64. The tab 80 is structured to engage the forward edge of the scalpel base wall 16 without extending into the path of the blade assembly 14. As with the post 76 of the third style of trap 70C, the tab 80 prevents the scalpel 10 from sliding forward in the jacket 50.

As shown in FIG. 4, a fifth type of trap 70E includes a wedge 82 and a rigid band 84. The wedge 82 is disposed on the outer side of at least one jacket sidewall 60, 62. The band 84 is a hoop structured to fit snugly about the jacket channel portion 54 and structured to move between a first, rearward position and a second, forward position. When a scalpel 10 is disposed in the jacket channel portion 54, the band 84 is moved to the forward position where the band 84 is brought into engagement with the wedge 82, the interaction between the wedge 82 and the band 84 causes the jacket sidewall 60, 62 to flex inwardly and be biased against the scalpel 10. The wedge 82 is disposed toward the back end of the jacket channel 64 so that the band 84 does not interfere with the scalpel slider 32. Thus, this type of trap 70E may only be used with scalpels 10 having a scalpel handle portion 28. Additionally, the band 84 further traps the scalpel 10 in the jacket channel 64 by preventing the scalpel 10 from lifting or pivoting out of the jacket channel 64. That is, when the band 84 is in the forward position engaging the wedge 82, the band 84 extends across the scalpel handle portion 28. A stop tab 86 may extend from the front side of the jacket body 52 to prevent the band 84 from slipping off the jacket body 52.

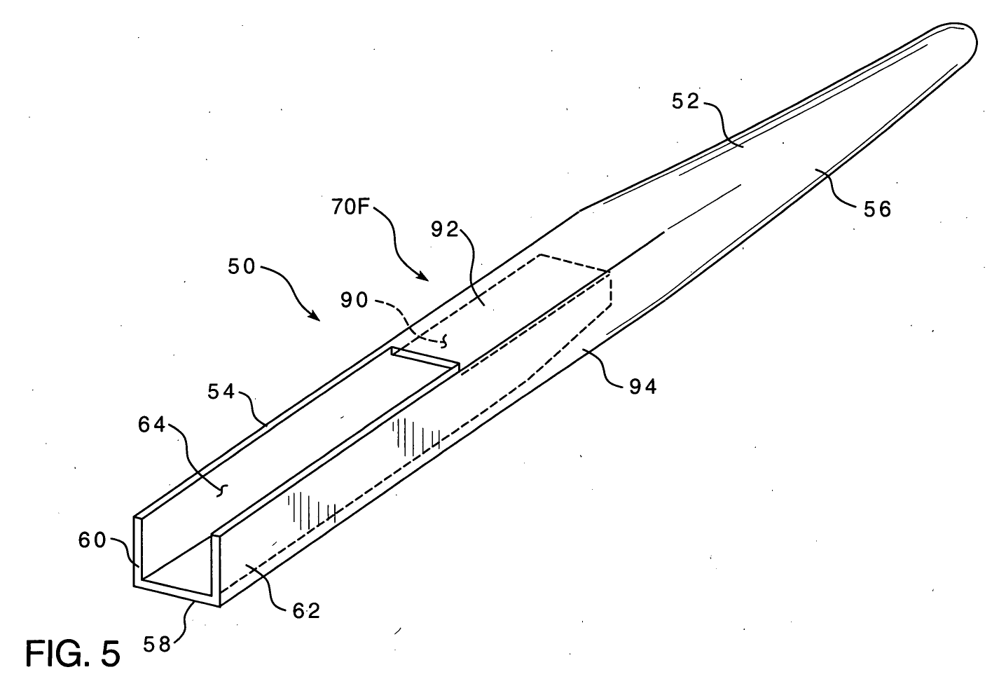

As shown in FIG. 5, a sixth type of trap 70F includes a pocket 90 formed by a ledge 92 extending from the jacket handle portion 56 over the rearward portion of the jacket channel 64. Similar to the fifth type of trap 70E described above, the ledge 92 does not extend so far as to interfere with the scalpel slider 32. Thus, this type of trap 70F may only be used with scalpels 10 having a scalpel handle portion 28. The pocket 90 prevents the scalpel 10 from lifting or pivoting out of the jacket channel 64. Therefore, this type of trap 70F is generally used in conjunction with one of the traps 70A, 70B, 70C shown in FIG. I that resist the scalpel 10 sliding axially. Additionally, or in the alternative, the pocket 90 may include a wedge portion 94. The wedge portion 94 extends upwardly from the jacket base wall 58 within the pocket 90. Thus, when a scalpel 10 is disposed in the pocket 90, the wedge portion 94 biases the scalpel against the ledge 92.

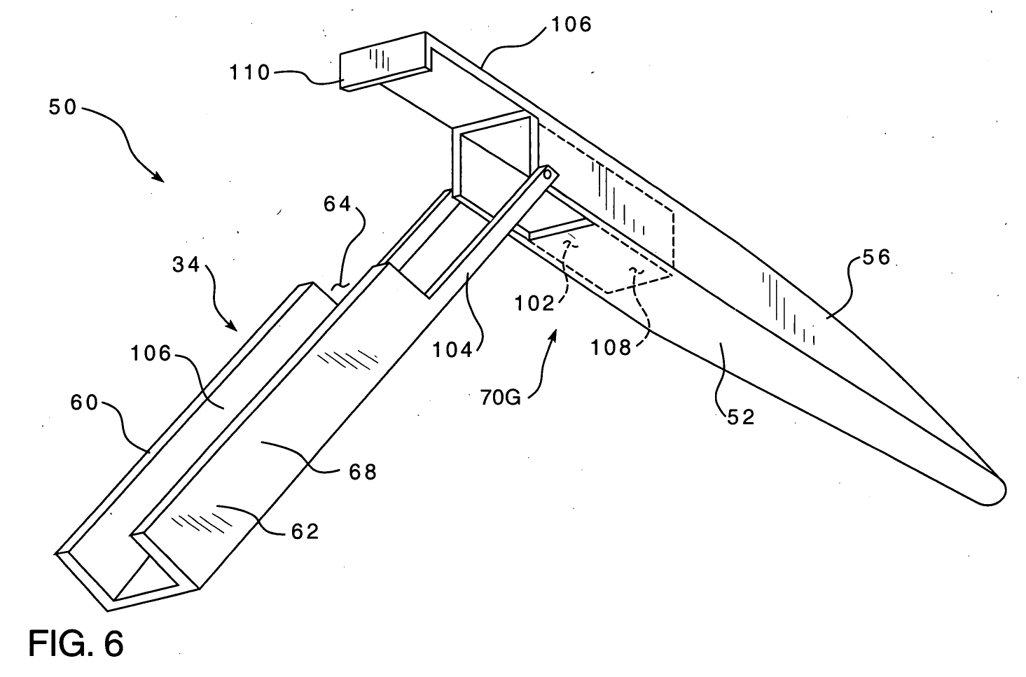

As shown in FIG. 6, a seventh type of trap 70G includes a bifurcated jacket channel portion 54 having a forward, pivoting portion 100 and a pocket portion 102. The forward, pivoting portion 100 includes the forward portion of the base wall 58 and the two sidewalls 60, 62 as well as a yoke 104. The pocket portion 102 is joined with the jacket handle portion 56 and includes the back portion of the base wall 58 and the two sidewalls 60, 62 as well as a ledge 106 extending from the jacket handle portion 56 over the rearward portion of the jacket channel 64. As the ledge 106 extends over the jacket channel 64 a pocket 108 is formed. The ledge 106 may include a tab 110 extending toward the base wall 58. The ledge tab 110 is structured to engage the back edge of the scalpel channel 26. The yoke 104 is pivotally coupled to the base wall 58 adjacent to the pocket 108, thereby pivotally coupling the pivoting portion 100 and a pocket portion 102. Thus, the pivoting portion 100 and a pocket portion 102 may pivot between a first, open and angled position (as shown) and a second, closed and generally linear position. This embodiment is functionally similar to the embodiment shown in FIG. 5, however, the pivoting portion 100 allows for easy insertion of the scalpel 10. As is known in the art, the pivoting portion 100 and a pocket portion 102 may include a snap-close structure, such as, but not limited to, a ball and detent (not shown) to hold the pivoting portion 100 and a pocket portion 102 in the closed position.

As shown in FIGS. 7 and 8, a scalpel assembly 150 includes a jacket 50 that is structured to engage a modified scalpel 10C. That is, both the jacket 50 and the modified scalpel 10C includes a portion of the trap 70H. The modified scalpel 10C is substantially similar to the scalpel described above with respect to the channel 26 and the blade assembly 14, and like reference numbers are used, however, the modified scalpel 10C further includes a scalpel trap portion 40 with at least one, and preferably two, passages 41 extending longitudinally therein. The passages 41 extend from the rear end of the scalpel 10C toward the channel 26. Adjacent to the channel 26 is a notch 42 associated with each passage. Each notch 42 has a back edge 44. The jacket 50 includes a jacket trap portion 46 structured to engage the scalpel trap portion 40. The jacket trap portion 46 includes at least one, and preferably two, arms 47 extending from the jacket handle portion 56 into the jacket channel portion 54. There is one arm 47 for each passage 41 in the scalpel handle 28. Each arm 47 has a distal tip 48 that turns outwardly. Further, the jacket sidewalls 60, 62 each have a cutout 49 disposed adjacent to the each tip 48. Thus, in operation, the user inserts the scalpel 10C into the jacket channel portion 54 with the rear end of the scalpel 10C in front of the scalpel trap portion 40. The user then slides the scalpel 10C longitudinally and rearward in the jacket 50 until the arms 47 move into the passages 41 and the tips 48 extend into the notches 42. The outwardly bent tips 48 engage the back edge 44 of the notch 42 to trap the scalpel 10C in the jacket 50. To release the scalpel 10C, the user applies an inward pressure to the tips 48 causing the arms 47 to flex until the arms 47 can be moved into the associated passage 41. At this point the user may pull the scalpel 10C in the longitudinal forward direction to remove the scalpel 10C. Thus, the scalpel trap portion 40 is structured to engage the jacket trap portion 46 to secure the scalpel 10C in the jacket 50.

The jacket 50 is, preferably, made from a heavy material such as, but not limited to a metal selected from the group consisting of: stainless steel, copper, brass, silver, or gold. The thickness of the material forming the jacket body 52 may be adjusted, e.g., be thicker or thinner in selected locations, to change the total weight and/or the balance of the jacket 50. That is, the jacket 50 may be made to be front or back heavy, or evenly balanced. Alternatively, the jacket 50 may be made from plastic and include a weighted structure 120. The weighted structure 120 is, preferably, an elongated member 122 made from a metal selected from the group consisting of: stainless steel, copper, brass, silver, or gold. The member 122 is embedded within the plastic matrix forming the jacket 50. The member 122 may have a variable shape, e.g., thicker in the front, structured to modify the weight and balance of the jacket 50. In an alternate embodiment, shown in FIG. 3, the weighted structure 120 is a plurality of granular elements 124, such as metal spheres or sand, embedded in the plastic matrix of the jacket body 52. As with the member 122, the granular elements 124 may be deposited in selected locations to modify the weight and balance of the jacket 50. Additionally, the weighted structure 120 may be sized to have a selected weight, typically between about 50 and 800 grams. Thus, the jacket 50 may be adapted to have the weight and balance that a particular surgeon prefers.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims appended and any and all equivalents thereof.

What is claimed is:

1. A jacket for a scalpel, said scalpel having an elongated body with a base wall and two sidewalls forming a scalpel channel, and a blade assembly having a blade coupled to a slider, said slider structured to move said blade between a first, retracted position and second, extended position, said jacket comprising:
a body having an elongated base wall and two sidewalls;
each sidewall extending generally perpendicular to said jacket base wall, thereby defining a jacket channel;
said jacket sidewalls spaced apart a sufficient distance so that said scalpel may be disposed between said jacket sidewalls; and
a trap structured to hold said scalpel in said jacket channel.

2. The jacket for a scalpel of claim 1, wherein said scalpel includes a flared handle and said trap includes a shaped portion of said jacket channel, said shaped portion structured to fit tightly about said flared scalpel handle.

3. The jacket for a scalpel of claim 1, wherein said trap includes at least one protrusion extending from one jacket sidewall toward the opposite jacket sidewall.

4. The jacket for a scalpel of claim 3, wherein said protrusion is structured to flex and thereby is structured to bias said scalpel against the opposite sidewall.

5. The jacket for a scalpel of claim 1, wherein said scalpel includes an opening in said base wall and said trap includes a post structured to engage said scalpel base wall opening.

6. The jacket for a scalpel of claim 1, wherein said trap includes a tab extending generally perpendicularly into said jacket channel from said base wall.

7. The jacket for a scalpel of claim 1, wherein said trap includes:
a wedge disposed on the outer side of at least one jacket sidewall;
a movable, rigid band extending around said jacket body, said band structured to move between a first, rearward position and a second, forward position; and
wherein when a scalpel is placed in said jacket channel portion and said band is moved to the forward position, said band is brought into engagement with said wedge and wherein the interaction between said wedge and said band causes said jacket sidewall to flex inwardly and be biased against said scalpel.

8. The jacket for a scalpel of claim 1, wherein:
said jacket body includes a handle portion coupled to and having the same general cross-sectional area as said jacket channel portion; and
said trap includes a pocket formed by a ledge extending from said jacket handle portion over said jacket channel portion;

9. The jacket for a scalpel of claim 8, wherein said trap includes a wedge in said pocket extending from said jacket base wall and structured to bias said scalpel against said ledge.

10. The jacket for a scalpel of claim 1, wherein:
said jacket body is bifurcated having a pivoting portion and a pocket portion;
said pivoting portion forming said jacket channel and having a yoke;
said pocket portion having a handle portion forming a pocket; and
said trap includes a tab extending from said pocket toward said jacket base wall.

11. The jacket for a scalpel of claim 1, wherein said body is made of metal selected from the group consisting of stainless steel, copper, brass, silver, or gold.

12. The jacket for a scalpel of claim 1, wherein said body is made of plastic, said plastic enclosing a weighted structure.

13. The jacket for a scalpel of claim 12, wherein said weighted structure is plurality of granular elements disposed in said plastic body.

14. The jacket for a scalpel of claim 12, wherein said weighted structure is an elongated member.

15. The jacket for a scalpel of claim 1, wherein said jacket body is weight biased to one end.

16. The jacket for a scalpel of claim 15, wherein said jacket body is made of plastic, said plastic enclosing a weighted structure.

17. The jacket for a scalpel of claim 16, wherein said weighted structure is plurality of granular elements disposed in said plastic body.

18. The jacket for a scalpel of claim 16, wherein said weighted structure is an elongated member.

19. The jacket for a scalpel of claim 1, wherein said jacket body includes a handle portion extending along one end of said body, said body being longer than said scalpel.

20. A scalpel assembly comprising:
a scalpel having an elongated body with a base wall and two sidewalls forming a scalpel channel, a blade assembly having a blade coupled to a slider, said slider structured to move said blade between a first, retracted position and second, extended position, and a handle portion;
said scalpel handle portion having a scalpel trap portion;
a jacket having a body with an elongated base wall and two sidewalls, each sidewall extending generally perpendicular to said jacket base wall, thereby defining a jacket channel, said jacket sidewalls spaced apart a sufficient distance so that said scalpel may be disposed between said jacket sidewalls, and a handle portion;
said jacket handle portion having a jacket trap portion; and
said scalpel trap portion structured to engage said jacket trap portion to secure said scalpel in said jacket.

21. The scalpel assembly of claim 20 wherein:
said scalpel trap portion having a notch in said scalpel handle and at least one passage extending through said scalpel handle into said notch; and
said jacket trap portion having at least one arm extending from said jacket handle portion into said jacket channel and structured to pass through said scalpel handle passage and engage said notch.
